(19) United States
(12) Patent Application Publication
(10) Pub. No.: US 2008/0113005 A1
(43) Pub. Date: May 15, 2008

(54) Composition Comprising an Extract of Gramineae Plant for the Prevention and Treatment of Ischemic Diseases and Degenerative Brain Diseases and the Use Thereof

(75) Inventors: Jong Won Lee, Daegu (KR); Ki-Chul Hwang, Seoul (KR); Hyung Soo Han, Daegu (KR); Sun Ha Lim, Daegu (KR); Hyeong-Kyu Lee, Daejeon (KR); Sang Hyuk Lee, Gyeongsangbuk-do (KR); Kyu Won Kim, Seoul (KR); Hyung Oh Jun, Seoul (KR); Hyun Jeong Kim, Daegu (KR); In-Seon Lee, Daegu (KR); Chae Ha Yang, Daegu (KR)

Correspondence Address: KIRK HAHN, 14431 HOLT AVE, SANTA ANA, CA, 92705, US

(73) Assignees: HYPOXI CO., LTD., Daegu (KR); SUN MOK INSTITUTE EDUCATION FOUNDATION, Daegu (KR)

(21) Appl. No.: 11/813,285

(22) PCT Filed: Jan. 4, 2006
(86) PCT No.: PCT/KR06/00027

§ 371 Date: Jul. 2, 2007

(30) Foreign Application Priority Data
Jan. 4, 2005 (KR) 10-2005-0000330
Dec. 30, 2005 (KR) 10-2005-0135745

(51) Int. Cl. A61K 36/899; A61K 9/00; A61P 25/28

(52) U.S. Cl. 424/441; 424/750; 424/439

(57) ABSTRACT

The present invention relates to a composition comprising extracts of Gramineae plant that improves cell viability under hypoxic conditions by inhibiting apoptosis. Thus, the extract of Triticum aestivum L., one of the Gramineae plant, of the present invention, in particular, prevents damage of brain, heart and kidney in animal models of ischemic diseases, and it also improves memory in an animal model of Alzheimer's disease. Therefore, a composition comprising extracts of Gramineae can be used as therapeutic agents or health care foods for preventing and treating ischemic diseases and degenerative brain diseases.

### TECHNICAL FIELD

The present invention relates to a composition comprising an extract of Gramineae plant for preventing and treating ischemic diseases and degenerative brain diseases and the use thereof.

### BACKGROUND ART

Cardiovascular disease, the leading cause of death worldwide each year, comprises approximately 30% of the all death, of which cerebral and myocardial infarction occupies 75%. Cerebral and myocardial infarction, the two representative ischemic diseases, are developed by the death of tissues in the brain and heart, respectively. These occur when cerebral or coronary arteries supplying blood to the respective tissue are occluded by thrombus or embolus under the condition where the blood vessels are already narrowed by arteriosclerosis induced by various factors such as hypertension, hyperlipidemia, diabetes, smoking etc. The best way to reduce myocardial and cerebral infarction is to reperfuse the occluded arteries as soon as possible, for which thrombolytic agents have been used to dissolve thrombus or embolus. However, for the therapy to be effective, thrombolytic agents should be given before the ischemic tissues became dead, preferably within 3-6 hours of occlusion. Usually it is difficult for the patients to be treated within 6 hours of occlusion. Therefore, preventing the tissue damage until occluded arteries are reperfused at the hospital is another way to reduce cerebral and myocardial infarction. Because one cause of the cell death in cerebral and myocardial infarction is due to apoptosis (Crow M T et al., Circ. Res., 95(10), pp 957-970, 2004; Friedlander R M, N. Engl. J. Med., 348(14), pp 1365-1375, 2003), antiapoptotic agents can be used to prevent the ischemic diseases.

These antiapoptotic agents can also be used to prevent the damage of transplanted tissues in kidney transplantation and plastic surgery because the damage also occurs by apoptosis during ischemia followed by reperfusion (ischemia-reperfusion) (Daemen M A et al., Transplantation, 73(11), pp 1693-1700, 2002; Gastman B R et al., Plast. Reconstr. Surg., 111, pp 1481-1496, 2003). Also, when the heart beat was stopped for a cardiac surgery, myocardial injury can occur if oxygen supply with pump-oxygenator is less than the oxygen demand by heart, or brain damage can occur if insufficient supply of blood to brain occurs due to hypotension. For instance, heart failure caused by myocardial damage, and hemiplegia caused by brain damage may occur when blood supply is blocked during coronary artery bypass graft, and aneurysm surgery performed in brain arteries and aorta, etc. Statistically, it has been reported that the 3-16% patients treated with operative or interventional therapy for aortic aneurysm shows various side effects, such as ischemic heart disease, renal failure, paraplegia etc. Accordingly, those side effects could be reduced if antiapoptotic agents are given before the operation.

The cause of neuronal cell death by apoptosis during cerebral infarction has not been well clarified so far, however it has been known that transient cerebral ischemia followed by the blockade of oxygen and glucose supply to the brain causes to reduce ATP concentration in the neuronal cells. This condition induces excessive accumulation of glutamate outside the cells, and subsequent influx of the glutamate into the cells causes accumulation of intracellular calcium ions, resulting in the neural cell apoptosis (Kang T C et al., J. Neurocytol., 30(12), pp 945-955, 2001). This damage is aggravated by the reactive oxygen species that are generated by abrupt supply of oxygen when ischemia is followed by re-perfusion of blood supply (Won M H et al., Brain Res., 836(1-2), pp 70-78, 1999). These reactive oxygen species are also a cause of neuronal cell death by apoptosis for several degenerative brain diseases, such as Alzheimer's disease, amyotrophic lateral sclerosis (Sayer L M et al., Curr Med Chem, 8, pp 721-738, 2001). Therefore, antiapoptotic agents can also be used to treat degenerative brain diseases, as well as ischemic diseases.

One such an antiapoptotic agent that has been evaluated is minocycline, a tetracycline antibiotic. Using animal models, it was shown that minocycline was effective in treating many diseases caused by apoptosis, such ischemic diseases as cerebral infarction (Yrjanheikki J et al., Proc. Natl. Acad. Sci. USA, 96(23), pp 13496-13500, 1999), myocardial infarction (Scarabelli T M et al., J. Am. Coll. Cardiol., 43(5), pp 865-874, 2004) and ischemic acute renal failure (Wang J et al., J. Biol. Chem., 279(19), pp 19948-19954, 2004), and such degenerative brain diseases as Alzheimer's disease (Hunter C L, Eur. J. Neurosci., 19(12), pp 3305-3316, 2004), Parkinson's disease (Wu D C et al., J. Neurosci., 22(5), pp 1763-1771, 2002), amyotrophic lateral sclerosis (Zhu S et al., Nature, 417(6884), pp 74-78, 2002), Huntington's disease (Chen. M. et al., Nat. Med., 6(7), pp 797-801, 2000) and spinal cord injury (Teng Y D et al., Proc. Natl. Acad. Sci. USA, 101(9), pp 3071-3076, 2004).

Some inventors of the present invention also confirmed that tetracycline antibiotics improved cell viability under similar ischemic conditions used in this study (See Korean Patent Registration No. 0404134; U.S. Pat. Nos. 6,716,822 and 6,818,625). Moreover, aminoglycoside and quinolone antibiotics also improved cell viability under the same ischemic condition as was used for minocycline. G418 (geneticin), an amino-glycoside antibiotic, in particular, was effective in treating myocardial infarction (U.S. Pat. No. 6,716,822). From further experiments, it was also shown that G418 improved cell viability under ischemic condition by inhibiting apoptosis, and that G418 was also effective in treating cerebral infarction. In conclusion, any agents that are screened, having the same effect as G418 in improving cell viability, might be effective in treating ischemic diseases and degenerative brain diseases.

The inventors of the present invention applied the screening method described above to find antiapoptotic agents from edible parts of plants because edible parts of plants are usually less toxic than chemicals. The inventors have found that the extract of some Gramineae plants described below, improved cell viability under hypoxic condition, which was proven by various in vitro tests, such as Trypan blue exclusion assay, MTT assay and so on. One of Gramineae plant, Triticum aestivum L. also known as wheat was used as a model plant for the further experiments. The crude extract extracted from seeds of Triticum aestivum L., inhibited apoptosis under hypoxic condition, which was proven by DNA fragmentation assay. In addition, the crude extract and/or purified fraction of seeds of Triticum aestivum L., were effective in treating ischemic diseases such as myocardial infarction, cerebral infarction, and ischemic acute renal failure, and also effective in treating a degenerative brain disease, Alzheimer's disease, the efficacy of which was proven by various animal model tests.

Various kinds of Gramineae plant were tested in the present invention, and characteristics of their seeds such as a composition and a use as a medicament were described as follows;

The dry seed of Horden vulgare L. also named as barley, has been reported to comprise 60˜68% starch, 8˜12% pentosan, 4˜5% cellulose, 4% lignin, 7˜14% nitrate component, 2˜3% ether extracts, and 2˜3% ash etc. The seed contains phosphatidyl serine, phosphatidyl choline, phosphatidyl ethanolamine, phosphatidine acid, sterols, ester, glycoside, alantoin etc. In addition, major fatty acids of the seed are palmitic acid, stearic acid, oleic acid and linoleic acid (Chung B S and Shin M K HyangyakDaesacheon, p 218, Youngrimsa, Seoul, 1998).

Malt, sprouted and dried barley, has been reported to stimulate digestive activity and lower blood glucose level (Ahn D K, Illustrated Book of Korean Medicinal Herbs, 5^{th} ed, p 476, Kyo-Hak Publishing Co., Ltd Seoul, 2002).

Brown rice, an unhusked rice (Oryza sativa L.), contains 15.5% water, 7.4% protein, 3.0% fat, 71.8% sugar, 1.0% fiber, 1.3% ash ingredient, and 0.54 mg/100 g of vitamin B1.

The seed of Avena sativa L. has similar amino acid composition to Brown rice, and has a large quantity of vitamin B groups. It has been used in Korean Oriental Medicine as a purgative, and an agent to treat constipation and cancers.

The seed of Zea mays L. has been reported to comprise 61.2% starch, 4.75% protein such as albumin, globulin, glutelin and etc., 0.21% alkaloid, and different kinds of vitamins. And the seed oil thereof comprises unsaturated fatty acids including palmitic, stearic, oleic, and linoleic acid etc. (Chung B S and Shin M K, HyangyakDaesacheon, p 234, Youngrimsa, Seoul, 1998).

The seed of Sorghum bicolor MOENCH has been reported to comprise 76.5% starch and 8.5% protein as main ingredients, and used in Korean Oriental Medicine to treat stomachache and acute gastroenteritis (Ahn D K, Illustrated Book of Korean Medicinal Herbs, 5^{th} ed, p 441, Kyo-Hak Publishing Co., Ltd Seoul, 2002).

The seed of Coix lacryma-jobi var. mayuen STAPF has been reported to comprise 67.7% starch, 13.8% protein, 5.1% lipid and 0.7% cellulose, and to be effective on diuresis, urination and etc. (Ahn D K, Illustrated Book of Korean Medicinal Herbs, 5^{th} ed, p 405, Kyo-Hak Publishing Co., Ltd Seoul, 2002).

The threshed seed of Panicum miliaceum L. has been reported to comprise 59.65% starch, 2.86% inorganic matter, 5.07% fatty acid such as palmitic, carnauba, margarinic, oleic, linolic and isolinolic acid, etc., and proteins such as albumin, gluterin, prolamin, etc. (Chung B S and Shin M K, HyangyakDaesacheon, p 225-226, Youngrimsa, Seoul, 1998).

The threshed seed of Setaria italica Beauv. has been reported to comprise 63.27% starch, 1.41% fat, 2.48% total nitrogen, 2.41% protein, 3.15% ash, 2.03% reduced sugar. The hull thereof contains proteins such as glutelin, prolamin, globulin and etc. (Chung B S and Shin M K, HyangyakDaesacheon, p 229, Youngrimsa, 1998). Setaria italica Beauv. has been reported to be effective on nausea, infirmity, dyspepsia and diarrhea (Ahn D K, Illustrated Book of Korean Medicinal Herbs, 5^{th} ed, p 705, Kyo-Hak Publishing Co., Ltd Seoul, 2002).

The seed of Secale cereale L. has been reported to comprise 70% starch, 12% protein, 2% fat, 1.7% inorganic ingredient as main ingredients. The prolamin and glutenin comprise more than 40% of total protein content.

The seed of Triticum aestivum L., also named as wheat, has been reported to be composed of approximately 82% endosperm, 16% pericarp and 2% embryo bud. Endosperm contains starch and protein such as gliadin and glutenin. And pericarp contains cellulose, protein and ash. Finally, embryo bud contains vitamin E and fatty acid such as oleic acid, linoleic acid, palmitic acid. Triticum aestivum L. has been used in Korean Oriental Medicine as a tranquilizer and as an agent for treating fever, ataraxis, and bleeding. Floating wheat is obtained by collecting wheat that is floating on the water when immature wheat is dried, and put in water. (Ahn D K, Illustrated Book of Korean Medicinal Herbs, 5^{th} ed, p 727, Kyo-Hak Publishing Co., Ltd Seoul, 2002).

It has been reported that the composition comprising the seed of Triticum aestivum L. and other Chinese herbs together was effective in the treating chest pain, scapulalgia, and cardiac diseases (Korean Patent Publication No. 10-2000-0033287). However, it has not been reported or disclosed about therapeutic efficacy of an extract of Gramineae plants as a major ingredient on the ischemic diseases and degenerative brain diseases in any of literatures cited above, the disclosures of which are incorporated herein by reference.

These and other objects of the present invention will become apparent from the detailed disclosure of the present invention, provided hereinafter.

### DISCLOSURE OF INVENTION

### Technical Problem

One cause of ischemic and degenerative brain diseases is due to apoptosis of cells in the respective organs, in addition to necrosis. Therefore, a pharmaceutical composition that can inhibit apoptosis can be used to prevent and treat the ischemic and degenerative brain diseases. Thus, it is important to develop a pharmaceutical composition that is effective in preventing and treating ischemic and degenerative brain diseases by inhibiting apoptosis, and, at the same time, that does not have toxicity problems.

### Technical Solution

The present invention provides a pharmaceutical composition comprising pharmaceutically acceptable carriers or adjuvants and also comprising a crude extract or purified fraction of Gramineae plant as an active ingredient, which can prevent ischemic and degenerative brain diseases by inhibiting apoptosis.

The present invention also provides a use of the crude extract or the purified fraction of Gramineae plant described above for the manufacture of a pharmaceutical composition, which can prevent ischemic and degenerative brain diseases by inhibiting apoptosis.

The present invention also provides a health care food or food additives comprising the crude extract or the purified fraction of Gramineae plant described above for the prevention or improvement of ischemic diseases and degenerative brain diseases by inhibiting apoptosis.

The term “ischemic diseases” disclosed herein comprise various ischemic diseases such as myocardial infarction, cerebral infarction, ischemic acute renal failure, ischemic acute hepatic failure, diabetic foot ulcer, diabetic nephropathy, and ischemic diseases or organ/tissue injury occurred by side effects of surgical operation.

The term “ischemic disease occurred by the side effect of surgical operation” disclosed herein comprises ischemic heart failure, ischemic renal failure, ischemic hepatic failure or ischemic stroke.

The term “organ/tissue injury occurred by the side effect of surgical operation” disclosed herein means the injury caused by ischemia-reperfusion when organ surgery, organ transplantation, or reconnection of parts of body severed by an accident is performed.

The term “organ/tissue” described above comprises internal organs, such as kidney, liver, pancreas, lung or heart, and external organs, such as leg, hand, finger or ear.

The term “degenerative brain diseases” disclosed herein comprise various degenerative brain diseases such as dementia of the Alzheimer's type (Alzheimer's disease), vascular dementia, Parkinson's disease, amyotrophic lateral sclerosis, Huntington's disease, Pick's disease, Creutzfeldt-Jakob disease or spinal cord injury.

The term “Gramineae plant” disclosed herein comprises various Gramineae plants such as Triticum aestivum L., floating Triticum aestivum L., Secale cereale L., brown rice, Horden vulgare L., malt, Avena sativa L., Zea mays L., Sorghum bicolor MOENCH, Coix lacryma-jobi var. mayuen STAPF, Panicum miliaceum L. or Setaria italica Beauv.

The term “crude extract” disclosed herein comprises the extract prepared by extracting plant material with water, lower alcohol such as methanol, ethanol, or the mixture thereof, preferably water.

Also, the term “purified fraction” disclosed herein comprises the purified fractions isolated from Gramineae plant prepared by the processes comprising the following steps; in step 1, adjusting the crude extract of Gramineae plant to pH 7 and pH 12 by adding strong base such as sodium hydroxide, potassium hydroxide etc; in step 2, removing the non-polar substance in the crude extract of Gramineae plant by adding equivalent amount of non-polar solvent to the extract prepared in step 1, to obtain water-soluble extract thereof; in step 3, subjecting to extraction and fractionation of the water soluble extract prepared in step 2, by adding equivalent amount of lower alcohol such as butanol thereto to obtain 4 purified fractions of the present invention, i.e., butanol-soluble fraction, and water-soluble fraction isolated from the extract prepared in pH 12 (designated as H12Bu and H12WA respectively, hereinafter); butanol-soluble fraction, and water-soluble fraction isolated from the extract prepared in pH 7 (designated as H7Bu and H7WA respectively, hereinafter), of which H12Bu is preferred.

Hereinafter, the present invention is described in detail.

An invented crude extract or purified fraction of Gramineae plant can be prepared, in detail, by the following procedures,

First of all, the invented crude extract of Gramineae plant may be prepared by the following steps: for example, Triticum aestivum L., floating Triticum aestivum L., Secale cereale L., brown rice, Horden vulgare L., malt, Avena sativa L., Zea mays L., Sorghum bicolor MOENCH, Coix lacryma-jobi var. mayuen STAPF, Panicum miliaceum L. or Setaria italica Beauv. is dried, cut, crushed and mixed with 1 to 15-fold, preferably, approximately 5- to 10-fold volume of distilled water, lower alcohols such as methanol, ethanol, butanol and the like, or the mixtures of any combination of two solvents with the ratio of about 1:0.1 10, preferably, water as one solvent; the solution that contains raw material to be extracted is treated with hot water at the temperature ranging from 20 to 100° C., preferably, from 50 to 100° C., for the period ranging from 0.5 to 48 hours, preferably, 1 to 24 hours, with the extraction method such as hot water extraction, cold water extraction, reflux extraction, or ultra-sonication extraction, with 1 to 12 times, preferably, hot water extraction with 3 to 4 times, consecutively; the extract is filtered and then the filtrate is concentrated with rotary evaporator, at the temperature ranging from 20 to 100° C., preferably, from 40 to 70° C., and then the concentrated filtrate is dried by vacuum freeze-drying, hot air-drying or spray drying to obtain dried crude extract powder of inventive crude extract of Gramineae plant which can be soluble in water, lower alcohols, or the mixtures thereof.

Secondly, inventive purified fraction of Gramineae plant may be prepared by the following steps: for example, the crude extract, preferably, the crude, water extract of Gramineae plant, prepared from the steps described above, is concentrated to about ⅕ to 1/20 of the original volume, preferably, approximately to 1/10 of the original volume to obtain the concentrated extract; then the concentrated extract is divided into two groups, of which one group is adjusted to pH 12 and the other one to pH 7, using strong base such as sodium hydroxide; then equivalent amount of ethyl acetate is added to each group, and the concentrated extract layer and the ethyl acetate layer in each group are mixed together vigorously and separated again to remove ethyl acetate layer and to recover the remaining, concentrated extract layer (water-soluble fraction) in each group; then the equivalent amount of water-saturated butanol is added to the water-soluble fraction of each group, and the water-soluble fraction and butanol layer in each group are mixed vigorously and separated again to recover both the water-soluble and butanol-soluble fractions in each group (i.e. pH 12 group and pH 7 group); and then the butanol-soluble fraction and the water-soluble fraction in pH 12 group are neutralized to pH 7 first, and then concentrated and dried to obtain H12Bu and H12WA fraction, respectively; and then the butanol-soluble fraction and the water-soluble fraction in pH 7 group are concentrated and dried to obtain H7Bu and H7WA fraction, respectively. Steps in concentration and drying, described above, involve concentration with rotary evaporator and drying with freeze dryer, respectively.

It is an object of the present invention to provide a use of a crude extract or purified fraction of Gramineae plant prepared by the methods described above, for the preparation of therapeutic agents for the prevention and treatment of ischemic and degenerative brain diseases in mammals and human, which were caused by apoptosis.

It is an object of the present invention to provide a method of preventing and treating ischemic and degenerative brain diseases in mammals and human, which were caused by apoptosis, comprising administrating an effective amount of a crude extract or purified fraction of Gramineae plant prepared by the methods described above, together with a pharmaceutically acceptable carrier thereof.

The activity of the crude extract or purified fraction of Gramineae plant prepared by the procedures described above, was tested by in vitro and in vivo experiments; in in vitro experiments, the ability of the crude extract of Gramineae plants to improve human hepatocellular carcinoma cells under hypoxic condition was shown by Trypan blue assay and MTT assay, and improvement of the cells by the extract under hypoxic condition occurred through inhibition of cell apoptosis, which was shown by DNA fragmentation assay. In addition, the ability of the purified fraction of Triticum aestivum L., a Gramineae plant, was shown by MTT assay; in in vivo experiments, the ability of the crude extract and the purified fraction of Triticum aestivum L. to prevent myocardial infarction, cerebral infarction and ischemic acute renal failure was shown, using appropriate rat models of ischemic diseases, and the ability of the crude extract of Triticum aestivum L. to prevent memory loss in Alzheimer disease was shown, using beta amyloid-induced rat model. In addition, the inventive extract can be used for the preventive purpose of the diseases because it can be used safely for a long time.

Therefore, the crude extract or purified fraction of Gramineae plants, prepared by methods described above, can be used as an active ingredient in preparing a pharmaceutical composition to prevent and treat ischemic and degenerative brain diseases. The inventive composition may additionally comprise approprate carriers, adjuvants or diluents, conventionally used in the art. The appropriate carriers, adjuvants or diluents is not limited to a specific material, and can be chosen, according to the usage and application method. Appropriate diluents are listed in the written text of Remington's Pharmaceutical Science (Mack Publishing Co., Easton Pa.).

Hereinafter, the formulation methods that are merely exemplary are shown below, and, in no way, limit the invention.

The pharmaceutical composition of the present invention comprising the crude extract or purified fraction of Gramineae plants as an active ingredient can also contain pharmaceutically acceptable carriers, adjuvants or diluents, such as lactose, dextrose, sucrose, sorbitol, mannitol, xylitol, erythritol, maltitol, starches, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and mineral oil. The formulations may additionally include fillers, anti-agglutinating agents, lubricating agents, wetting agents, flavoring agents, emulsifiers, preservatives and the like.

The compositions of the invention may be formulated so as to provide quick, sustained or delayed release of the active ingredient after their administration to a patient by employing any of the procedures well known in the art. For example, the compositions of the present invention can be dissolved in oils, propylene glycol, or other solvents that are commonly used to produce injectables.

Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing the present compositions, may be prepared in any form, such as oral dosage form (powder, tablet, capsule, soft capsule, aqueous medicine, syrup, elixir, pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the patients, severity of the diseases, drug form, route and period of administration, and may be chosen by those skilled in the art. However, in order to obtain desirable effects, it is generally recommended to administer at the amount ranging 10 mg/kg, preferably, 0.1 to 1000 mg/kg by weight/day of the inventive extract or composition of the present invention. The dose may be administered in single or divided into several times per day.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Also, the present invention provides a health care food comprising the crude extract or purified fraction of Gramineae plant as an active ingredient, together with a sitologically acceptable additives for the prevention and improvement of ischemic diseases or degenerative brain diseases caused by apoptosis.

The term “health care food” disclosed herein comprises dietary supplements, nutraceuticals, food or food additives.

The health care food of the present invention comprises 0.01 to 95% (preferably 1 to 80%) of the above crude extract or purified fraction by weight, based on the total weight of the composition.

Health care food described above comprises health functional food, and health beverage, etc., and may be used as powder, granule, tablet, chewing tablet, capsule, beverage, etc. The health functional and health beverage that contain the extract of Gramineae plant described above can be used for the prevention and improvement of ischemic diseases and degenerative brain diseases.

The health beverage may comprise, in general, 0.02 to 5 g (preferably 0.3 to 1 g) of the above crude extract or purified fraction per 100 ml of the health beverage composition. In addition to the crude extract at the specified ratio, the health beverage composition of present invention may contain, without any limitation, various flavoring agents or natural carbohydrates, as was found in conventional beverages. Examples of the aforementioned flavoring agents are natural flavorings such as taumatin, stevia extract (levaudioside A, glycyrrhizin et al.), and synthetic flavoring agents such as saccharin, aspartam, etc. Examples of the aforementioned natural carbohydrates are monosaccharide such as glucose, fructose etc; disaccharides such as maltose, sucrose etc; conventional sugars such as dextrin, cyclodextrin; and sugar alcohols such as xylitol, and erythritol etc. The health beverage may comprise, in general, about 1 to 20 g (preferably 5 to 12 g) of the above natural carbohydrates per 100 ml of the health beverage composition.

In addition to the components mentioned above, the composition of the present invention may also contain various nutrients, vitamins, minerals (electrolytes), synthetic and natural flavoring agents, coloring agents, improving agents (such as cheese and chocolate, etc.), pectic acids and the salt thereof, alginic acids and the salt thereof, organic acids, protective colloidal adhesives, pH controlling agents, stabilizers, preservatives, glycerin, alcohols, and carbonizing agent used in carbonate beverage, etc. The composition of the present invention may also contain the pulp to manufacture natural fruit juice, fruit juice beverage and vegetable beverage, wherein the component can be used independently or in combination. The ratio of the components is not so important, but is generally in the range of about 0 to 20 w/w % per 100 w/w % of present composition.

Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as sodium phosphate, potassium phosphate, acid pyrophosphate, polyphosphate; natural anti-oxidants, such as polyphenol, catechin, tocopherol, rosemary extract, vitamin C, green tea extract, licorice root extract, chitosan, tannic acid, phytic acid, etc.

The above-described inventive extract may be 20 to 90% high concentrated liquid, power, or granule type.

Similarly, the above-described inventive extract can comprise additionally one or more than one of lactose, casein, dextrose, glucose, sucrose and sorbitol.

Inventive extract of the present invention have no toxicity and adverse effect therefore; they can be used safely.

It will be apparent to those skilled in the art that various modifications and variations can be made in the compositions, use and preparations of the present invention without departing from the spirit or scope of the invention.

### Advantageous Effects

The extracts of Gramineae plant of the present invention reduced the infarcted volume for animal models of ischemic diseases, and also prevented brain damage, resulting in the enhancement of memory for an animal model of Alzheimer's disease, both by inhibiting apoptosis. Because the extracts have no side effect even if they are taken for a long time, they can be used as a therapeutics agent or health care food for preventing and treating ischemic or degenerative brain diseases.

### BRIEF DESCRIPTION OF THE DRAWINGS

The above and other objects, features and other advantages of the present invention will be understood more clearly from the following detailed description taken in conjunction with the accompanying drawings, in which;

FIG. 1 shows improvement of viability of HepG2 cells under hypoxic condition by addition of different concentrations of the crude extract of Triticum aestivum L. into the growth medium.

FIG. 2 shows effect of different concentrations of the crude extract of Triticum aestivum L. on the viability of HepG2 cells under normoxic condition.

FIG. 3 shows improvement of viability of HepG2 cells under hypoxic condition by addition of different concentrations of the crude extract of Gramineae plants (brown rice, Secale cereale L., malt, Triticum aestivum L. and floating Triticum aestivum L.) into the growth medium, measured by MTT assay.

FIG. 4 shows improvement of viability of HepG2 cells under hypoxic condition by addition of different concentrations of the crude extract of Gramineae plants (Coix lacryma-jobi var. mayuen STAPF, Zea mays L., Sorghum bicolor MOENCH, Panicum miliaceum L., Horden vulgare L., Avena sativa L., Setaria italica Beauv., Secale cereale L. and Triticum aestivum L. into the growth medium, measured by MTT assay.

FIG. 5 shows improvement of viability of HepG2 cells under hypoxic condition by addition of 400 μg/ml of the crude extract of Triticum aestivum L. into the growth medium.

FIG. 6 shows patterns of DNA fragmentation of HepG2 cells under hypoxic condition.

FIG. 7 shows the inhibitory effect of the crude extract of Triticum aestivum L. on DNA fragmentation of HepG2 cells under hypoxic condition.

FIG. 8 shows improvement of viability of HepG2 cells under hypoxic condition by addition of different concentrations of butanol-soluble and water-soluble fraction of Triticum aestivum L. into the growth medium, measured by MTT assay.

FIG. 9 shows inhibitory effect of the crude extract of Triticum aestivum L. administered intraperitoneally on infarcted volume for an animal model of myocardial infarction.

FIG. 10 presents an example of a section of infarcted heart, stained with TTC (2,3,5-triphenyltetrazolium chloride), when physiological saline solution was administered intraperitoneally.

FIG. 11 presents an example of a section of infarcted heart, stained with TTC (2,3,5-triphenyltetrazolium chloride), when the crude extract of Triticum aestivum L. was administered intraperitoneally.

FIG. 12 shows inhibitory effect of the crude extract of Triticun aestivum L. administered orally on infarcted volume for an animal model of myocardial infarction.

FIG. 13 presents an example of a thin section of infarcted heart, stained with Hematoxylin & Eosin, when physiological saline solution was administered orally.

FIG. 14 presents an example of a thin section of infarcted heart, stained with Hematoxylin & Eosin, when the crude extract of Triticum aestivum L. was administered orally.

FIG. 15 shows inhibitory effect of the crude extract of Triticum aestivum L. administered orally on infarcted volume for an animal model of cerebral infarction.

FIG. 16 presents an example of a section of infarcted brain, stained with TTC (2,3,5-triphenyltetrazolium chloride), when physiological saline solution was administered orally.

FIG. 17 presents an example of a section of infarcted brain, stained with TTC (2,3,5-triphenyltetrazolium chloride), when the crude extract of Triticum aestivum L. was administered orally.

FIG. 18 shows inhibitory effect of the crude extract of Triticum aestivum L. administered intraperitoneally on renal injury for an animal model of ischemic acute renal failure, by measuring serum creatinine levels.

FIG. 19 shows inhibitory effect of butanol-soluble fraction of Triticum aestivum L. administered intraperitoneally on renal injury for an animal model of ischemic acute renal failure, by measuring serum creatinine levels.

FIG. 20 shows inhibitory effect of the crude extract of Triticum aestivum L. administered orally on the loss of memory for an animal model of Alzheimer's disease, measured using water-maze test.

FIG. 21 shows improving effect of the crude extract of Triticum aestivum L. administered orally on memory for an animal model of Alzheimer's disease, measured using water-maze test.

### BEST MODE FOR CARRYING OUT THE INVENTION

It will be apparent to those skilled in the field that various modifications and variations can be made in the compositions, use and preparations of the present invention without departing from the aim and scope of the invention.

The present invention is more specifically explained by the following examples. However, it should be understood that the present invention is not limited to these examples in any manner.

The following Reference Example, Examples and Experimental Examples are intended to further illustrate the present invention without limiting its scope.

### EXAMPLE 1

### The Preparation of Crude Extracts of the Gramineae Plants

100 g of Triticum aestivum L. purchased from the market was washed, mixed with 2 L of water and extracted two times, using electric brewing pot for oriental herb medicine (Daewoong oriental herb medicine electric brewing pot DWP-2000, Daewoong). The extract was filtered to obtain 2 L of filtered extract, and the extract was lyophilized to obtain 22 g of dried crude extract of Triticum aestivum L., which is named as HY6228Hereinafter.

When the same extraction method described above, was applied to other herbs, 8, 6, 11, 18, 20, 44, 9, 10, 14, 32 and 6 g per 100 g of crude extract of Secale cereale L., Horden vulgare L., malt, floating Triticum aestivum L., brown rice, Avena sativa L., Zea mays L., Sorghum bicolor MOENCH, Coix lacryma-jobi var. mayuen STAPF, Panicum miliaceum L. and Setaria italica Beauv were obtained, respectively, and each of the extract was named as HY6228B, HY6228C, HY6113, HY6138, HY6228A, HY6228I, HY6228F, HY6228E, HY6228G, HY6228D and, HY6228H hereinafter, respectively.

### EXAMPLE 2

### The Preparation of Purified Fractions of the Gramineae Plants

2 L of the crude extract of Triticum aestivum L. prepared in Example 1 was concentrated to 200 ml ( 1/10 volume of crude extract) by rotary evaporator under reduced pressure. The concentrated sample was divided into two groups, and both groups were adjusted to pH 12 and pH 7, using sodium hydroxide. Each of the adjusted samples prepared above was mixed with equivalent volume of ethyl acetate vigorously, and divided into ethyl acetate fractions and water-soluble fractions. Water-soluble fractions were collected and mixed with equivalent volume of butanol vigorously, and separated into butanol fraction and water-soluble fraction. Identical procedure with above-described steps was repeated 3 times to collect 300 ml of butanol fractions respectively.

The butanol fraction and water-soluble fraction obtained from the extract adjusted to pH 12 were neutralized to pH 7 and were concentrated to obtain 0.4 g of butanol soluble fraction (designated as H12Bu hereinafter) and 2 g of water-soluble fraction (designated as H12WA hereinafter) of Triticum aestivum L., respectively. The butanol fraction and water-soluble fraction obtained from the extract adjusted to pH 7 was concentrated directly to obtain 0.1 g of butanol soluble fraction (designated as H7Bu hereinafter) and 2 g of water-soluble fraction (designated as H7WA hereinafter) of Triticum aestivum L., respectively.

### REFERENCE EXAMPLE 1

### Experimental Animal

Male Sprague-Dawley rats (Hyochang Science, Korea) weighing 250-300 g were bred at temperature (21±1° C.) and in a 12-h light/dark cycle with food and water ad libitum. Prior to experiments, experimental animals were handled for 10 minutes.

### EXPERIMENTAL EXAMPLE 1

### Measurement of Improving Effect of Crude Extracts of Gramineae Plant on Cell Viability (in vitro)

To determine the improving effect of the crude extract of Gramineae plant on HepG2 cell viability at hypoxia or normoxia, viable cell numbers under hypoxic and normoxic conditions were measured at various HY6228 concentrations in the medium, in accordance with modified trypan blue dye-exclusion assay which is described in the literature (Sambrook J and Russel D W, Molecular Cloning 3rd ed, Vol. 3, A8.6-8.8, Cold Spring Harbor Laboratory Press, New York, 2001).

HepG2 cells (human hepatoma cell line, ATCC HB 8065, 2×10⁵ cells/800 μl) were seeded onto each well of 12-well plate and incubated in EMEM (Eagle's minimum essential medium; Invitrogen, USA), supplemented with penicillin G sodium (100 Units/L, Invitrogen, USA), streptomycin sulfate (100 mg/L, Invitrogen, USA) and 10% (w/v) fetal bovine serum (Invitrogen, USA) at 37° C. for 48 hours in 5% CO₂-95% air incubator. After the media was changed with fresh medium, the number of viable cells was measured for 2 days under hypoxic (3% of oxygen concentration) and normoxic conditions at the concentrations of 0 (negative control), 100, 1000 μg/ml of HY6228 dissolved in 50% ethanol.

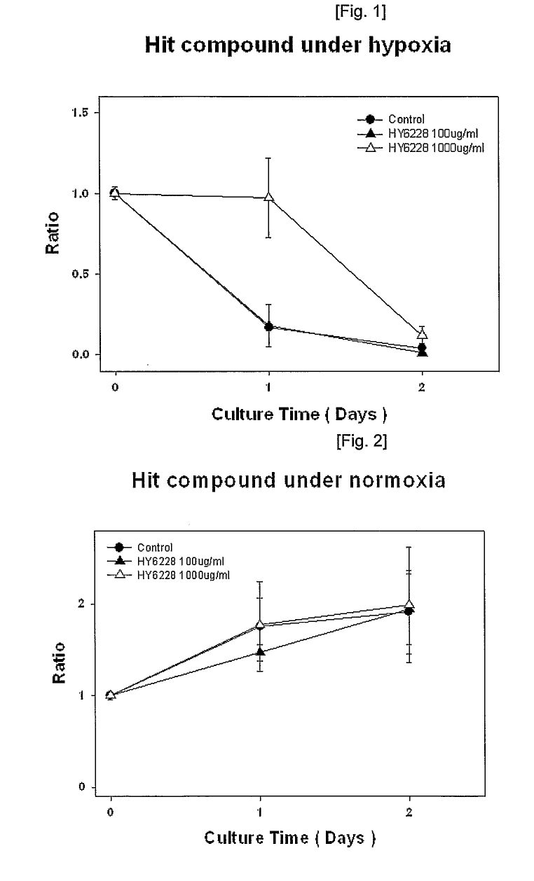

Media was removed, and the cells were washed once with PBS solution (phosphate buffered saline) and then trypsinized. The cells were harvested with centrifugation and resuspended in fresh media to form cell suspension. The suspension was mixed with equal volume of 0.4% trypan blue solution (Invitrogen, USA). Five minutes later, the number of the viable cells was counted with hemocytometer by regarding cells stained with blue color as dead cells, and unstained cells as viable cells, respectively. As shown in FIG. 1, one day after incubation under hypoxic condition, most of cells were dead (Ratio=0) for negative control group (Control), while most of cells were still alive for the cells treated with the invented extract (HY6228) at 1000 μg/ml (Ratio>0). As shown in FIG. 2, growth rate of cells treated with 1000 μg/ml HY6228 was similar to that of control group under normoxic condition. The results show that HY6228 improves cell survival significantly under hypoxic condition without inhibiting cell growth under normoxic condition, even at the concentration of 1000 μg/ml.

To investigate the improving effect of HY6228A, HY6228B, HY6228C, HY6113, HY6138, HY6228I, HY6228F, HY6228E, HY6228G, HY6228H and HY6228D, prepared in Example 1, on HepG2 cell survival in medium under hypoxic condition, MTT assay was performed as described previously (Hoffman R M, In Cell Biology (Celis J E (Ed), Vol. 1, pp 369-370, Academic Press, New York, 1994), with minor modification.

HepG2 cells (human hepatoma cell line, ATCC HB 8065, 2×10⁵ cells/800 μl) were seeded onto each well of 12-well plate and incubated in EMEM (Eagle's minimum essential medium; Invitrogen, USA), supplemented with penicillin G sodium (100 Units/L, Invitrogen, USA), streptomycin sulfate (100 mg/L, Invitrogen, USA) and 10% (w/v) fetal bovine serum (Invitrogen, USA) at 37° C. for 48 hours in 5% CO₂-95% air incubator. Then 1000 μg/ml of crude extract of each HY6228A, HY6228B, HY6113, HY6138 and HY6228, prepared in example 1 and dissolved in 50% ethanol, was added to the medium. At the same time, 10 μg/ml of G418 (Invitrogen, USA) that has been shown to improve cell viability, was also added separately to use as a positive control (Positive). Forty-eight hours of incubation under hypoxic condition, cell viability was measured by MTT assay (FIG. 3).

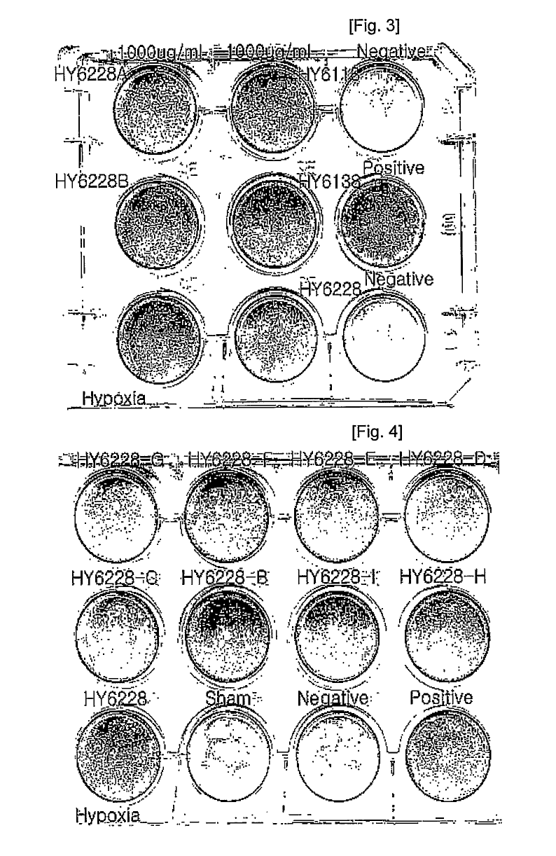

In addition, 1000 μg/ml of crude extract of each HY6228C, HY6228D, HY6228E, HY6228F, HY6228G, HY6228H and HY6228I, prepared in example 1, dissolved in 50% ethanol/50% glycerol and diluted to a final concentration of 0.5% ethanol/0.5% glycerol, was also added to the medium. Forty-eight hours of incubation under hypoxic condition, cell viability was measured by MTT assay (FIG. 4).

As shown in FIG. 3, the crude extracts of brown rice (HY6228A), Secale cereale L. (HY6228B), malt (HY6113) and floating Triticum aestivum L. (HY6138) improved cell viability under hypoxic condition as much as crude extract of Triticum aestivum L. (HY6228) did.

As shown in FIG. 4, the crude extracts of Coix lacryma-jobi var. mayuen STAPF HY6228G), Zea mays L. (HY6228F), Sorghum bicolor MOENCH (HY6228E), Panicum miliaceum L. (HY6228D), Horden vulgare L. (HY6228C), Avena sativa L. (HY6228I) and Setaria italica Beauv. (HY6228H) also strongly improved cell viability under hypoxic condition.

The crude extracts of Triticum aestivum L. (HY6228) and Secale cereale L. (HY6228B) improved cell viability the most among those invented crude extracts.

In summary, it can be concluded if the crude extract of Triticum aestivum L. can prevent and treat ischemic diseases and degenerative brain diseases in animal model, the crude extracts of Secale cereale L., brown rice, malt, floating Triticum aestivum L., Coix lacryma-jobi var. mayuen STAPF, Zea mays L., Sorghum bicolor MOENCH, Panicum miliaceum L., Horden vulgare L., Avena sativa L. and Setaria italica Beauv., which belong to the same Gramineae family as Triticum aestivum L., will show similar efficacy to the diseases.

### EXPERIMENTAL EXAMPLE 2

### Inhibitory Effect of Crude Extract of Triticum aestivum L. on the Apoptosis Under Hypoxic Condition (in vitro)

To identify a mechanism how HY6228 improves cell survival under hypoxic condition, DNA fragmentation assay was performed in accordance with the procedure disclosed in cited literature with minor modification (Yoshida A et al., In Apoptosis: A practical approach, Studzinski GP (Ed), pp 47-48, Oxford University Press, New York, 1999).

HepG2 cells (human hepatoma cell line, ATCC HB 8065, 1×10⁶ cells/4 ml) were seeded onto 60 mm dish and incubated in EMEM (Eagle's minimum essential medium; Invitrogen, USA), supplemented with penicillin G sodium (100 Units/L, Invitrogen, USA), streptomycin sulfate (100 mg/L, Invitrogen, USA) and 10% (w/v) fetal bovine serum (Invitrogen, USA) at 37° C. in 5% CO₂-95% air incubator for 48 hours. After the media was changed with fresh media, DNA fragmentation assay was performed for 2 days under hypoxic (3% of oxygen concentration) and normoxic conditions at the concentrations of 0 (negative control) and 400 μg/ml of HY6228, prepared in Example 1 and dissolved in 50% ethanol.

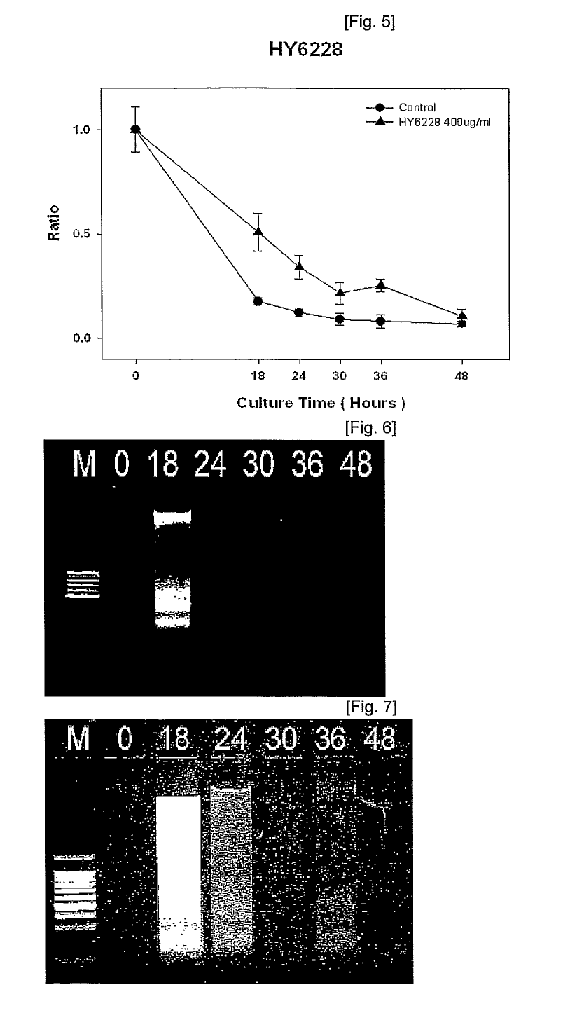

As the result, HY6228 effectively improved cell viability (Refer to FIG. 5) and retarded appearance of DNA ladder (FIG. 7), compared with negative control (Refer to FIG. 6). Therefore, we can conclude that HY6228 improves cell viability under hypoxic condition by inhibiting apoptosis

### EXPERIMENTAL EXAMPLE 3

### Improving Activity on Cell Viability of the Purified Fraction Isolated from Triticum aestivum L. (in vitro)

To investigate effect of the purified fractions isolated from HY6228 prepared in Example 2, MTT assay was performed in accordance with the procedure cited in the literature with minor modification (Hoffman R M, In Cell Biology (Celis J E (Ed), Vol. 1, pp 369-30, Academic Press, New York, 1994).

HepG2 cells (human hepatoma cell line, ATCC HB 8065, 2×10⁵ cells/800 μl) were seeded onto 12 well-plate and incubated in EMEM (Eagle's minimum essential medium; Invitrogen, USA), supplemented with penicillin G sodium (100 Units/L, Invitrogen, USA), streptomycin sulfate (100 mg/L, Invitrogen, USA) and 10% (w/v) fetal bovine serum (Invitrogen, USA) at 37° C. in 5% CO₂-95% air conditioned incubator for 48 hours. After changing media to fresh one, the cells were treated with medium containing 100 μg/ml or 1000 μg/ml of the purified fractions isolated from HY6228 prepared in Example 2 dissolved in DMSO (in case of butanol soluble fractions) or 50% ethanol (in case of water soluble fractions) as test groups and the group without addition was regarded as a negative control group. The group treated with 10 μg/ml of G418 (Invitrogen, USA) well known to improve cell viability was regarded as a positive control group. The groups were incubated at 37° C. in 5% CO₂-95% air-conditioned incubator for 48 hours, and the cells were subjected to MTT assay.

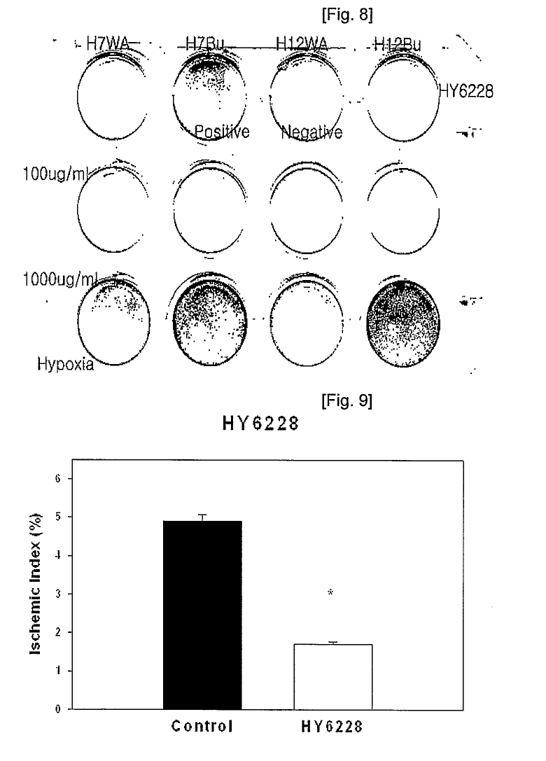

As shown in FIG. 8, the test group treated with 1000 μg/ml of H7WA and H7Bu prepared in example 2 slightly improved cell viability at hypoxic condition, and 1000 μg/ml of H12Bu prepared in Example 2 slightly improved cell viability at hypoxic condition, while 1000 μg/ml of H12WA prepared in Example 2 has no effect on cell viability. Therefore, it is confirmed that H12Bu fraction is enriched with an active ingredient showing potently improving effect on cell viability.

### EXPERIMENTAL EXAMPLE 4

### Effect of Crude Extract of Triticum aestivum L. Administered Intraperitoneally on Myocardial Infarction (in vivo)

The therapeutic efficacy of HY6228 administered intraperitoneally on myocardial infarction was determined, using animal model in accordance with the procedure cited in the literature (Haisong J et al., Circulation, 97, pp 892-899, 1998).

SD rats prepared as in Reference Example 1 were anesthetized with 10 mg/kg ketamine (Yuhan Corp., KOREA) and 5 mg/kg xylazine (Sigma, USA) and intubated endotracheally. After the thorax was opened by the excision of 3rd and 4th ribs, the heart was delivered from intercostal inwards. The left coronary artery was ligated with 5-0 prolene thread and the heart was rearranged in the thorax. The subcutaneous tissues and skin were sutured to complete myocardial infarction animal model.

HY6228 prepared in Example 1 was injected to myocardial infarct animal model and myocardial infarct size was determined. To determine the safe and effective dose of injection, the test was started from the minimum dose based on the result of Experimental Example 1, and the dose was gradually increased using dose-doubling method, for example, the dose was increased from 50, 100 to 200 mg/kg, etc in the order.

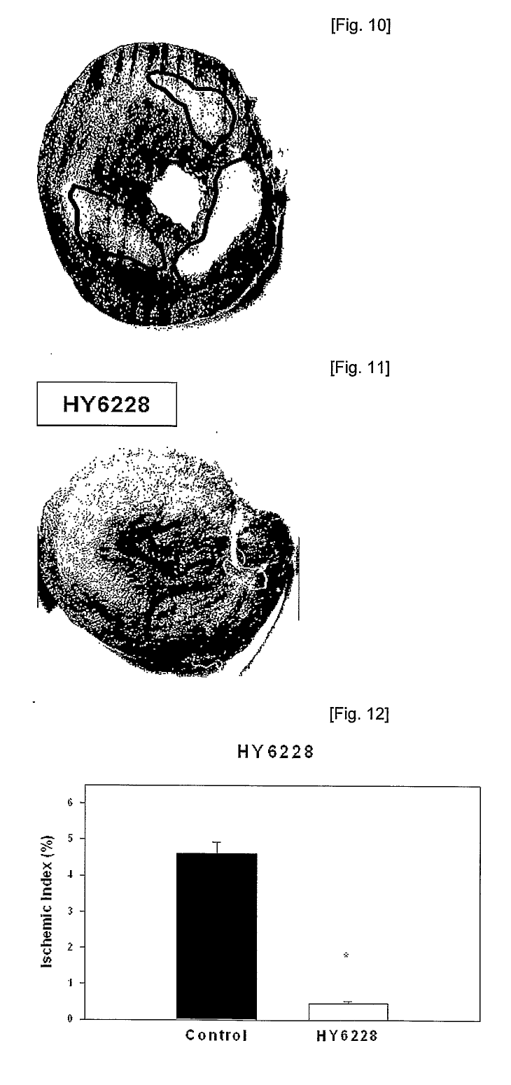

One hour prior to the left coronary artery ligation, 1 ml of HY6228 or vehicle was injected intraperitoneally at a dose of 400 mg/kg each. Three days after the ischemia, the delivered heart was stained with TTC (2,3,5-triphenyltetrazolium chloride, Sigma, USA) solution, and the infarct volume was determined by image analysis system (Qunatity One 4.2, Bio-Rad, USA). One example of sections of hearts, stained with TTC, for vehicle-treated group and HY6228-treated group are shown in FIGS. 10 and 11, respectively.

Efficacy was compared with Ischemic index (%), calculated as shown in Math FIG. 1.

$$IschemicIndex ( \% ) = \frac{A}{B} \times 100 \qquad MathFigure 1$$

A: Infarcted volume of the heart (mm³),
B: Total volume of the heart (mm³).

The ischemic index (%) of the HY6228-treated group (n=7) was 1.8%, while that of the vehicle-treated group (n=6) was 4.9%, indicating that HY6228 potently reduces the infarct size (63%, p<0.01) (FIG. 9). Therefore, HY6228 is effective in treating myocardial infarction when HY6228 is administered intraperitoneally.

### EXPERIMENTAL EXAMPLE 5

### Effect of Crude Extract of Triticum aestivum L. Administered Orally on Myocardial Infarction (in vivo)

The therapeutic efficacy of HY6228 administered orally on myocardial infarction was determined, using animal model in accordance with the procedure cited in the literature (Haisong J et al., Circulation, 97, pp 892-899, 1998).

SD rats prepared as in Reference Example 1 were fed on food mixed with HY6228 (400 mg/kg) for three days, and then ischemia was induced by the left coronary artery ligation. Three days after ischemia, the heart was delivered, and stained with TTC solution and the infarct volume was measured by image analytical system (Qunatity One 4.2, Bio-rad, USA). Ischemic index (%) was calculated according to the Math FIG. 1 to compare with the efficacy of HY6228. In addition, thin section of the heart was stained with Hematoxylin and Eosin (Sigman, USA), and the damage of the heart tissue was observed by microscope at the cellular level.

The ischemic index (%) of the HY6228-treated group (n=10) was 0.46%, while that of the vehicle-treated group (n=4) was 4.6%, indicating that HY6228 potently reduces the infarct size (90%, p<0.001) (FIG. 12). Also, there was a significant reduction in cell injury for HY6228-treated group (FIG. 14), compared with that for vehicle-treated group (FIG. 13) when the cells were stained with Hematoxylin and Eosin. Therefore, HY6228 is effective in treating myocardial infarction even when HY6228 is administered orally.

### EXPERIMENTAL EXAMPLE 6

### Effect of Crude Extract of Triticum aestivum L. Administered Orally on Cerebral Infarction (in vivo)

The therapeutic efficacy of HY6228 orally administered on cerebral infarction were determined, using animal model in accordance with the procedure cited in the literature (Han H S et al., J. Neurosci., 22, pp 3921-3928, 2002) with minor modification.

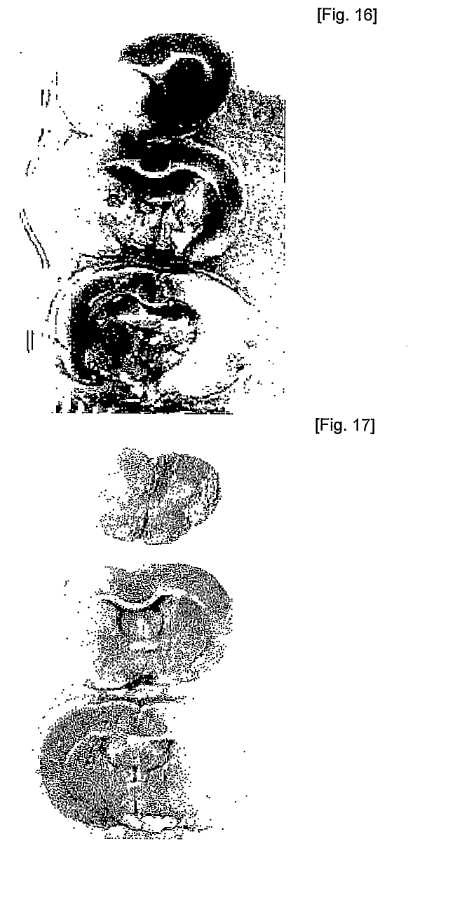

SD rats prepared 566 as in Reference Example 1 were anesthetized with enflurane (Choongwae Pharm. Corp., KOREA) by inhalation. The neck of rats was incised to expose carotid artery, and the carotid and the external carotid artery was ligated 3-0 nylon thread was inserted into the internal carotid artery, and ischemia was induced by blocking middle cerebral artery (MCA). Four-hundred mg/kg of HY6228 dissolved in 0.5 ml or the same volume of vehicle was orally administered daily from day seven to day one before ischemia was induced. Two hours after the ischemia, the MCA blood flow was recovered by removing the thread Twenty-two hours of reperfusion, the rats were induced euthanasia to deliver the brain and brain tissue was stained in TTC solution. One example of sections of brains, stained with TTC, for vehicle-treated group and HY6228-treated group are shown in FIGS. 16 and 17, respectively.

The infarct volume of the cerebral hemisphere was determined by image analysis system (Qunatity One 4.2, Bio-Rad, USA). Efficacy was compared with Infarct index (%), calculated as shown in Math FIG. 2.

$$IschemicIndex ( \% ) = \frac{A}{B} \times 100 \qquad MathFigure 2$$

A: Infarcted volume of the cerebral hemisphere (mm³),
B: The total volume of the cerebral hemisphere (mm³).

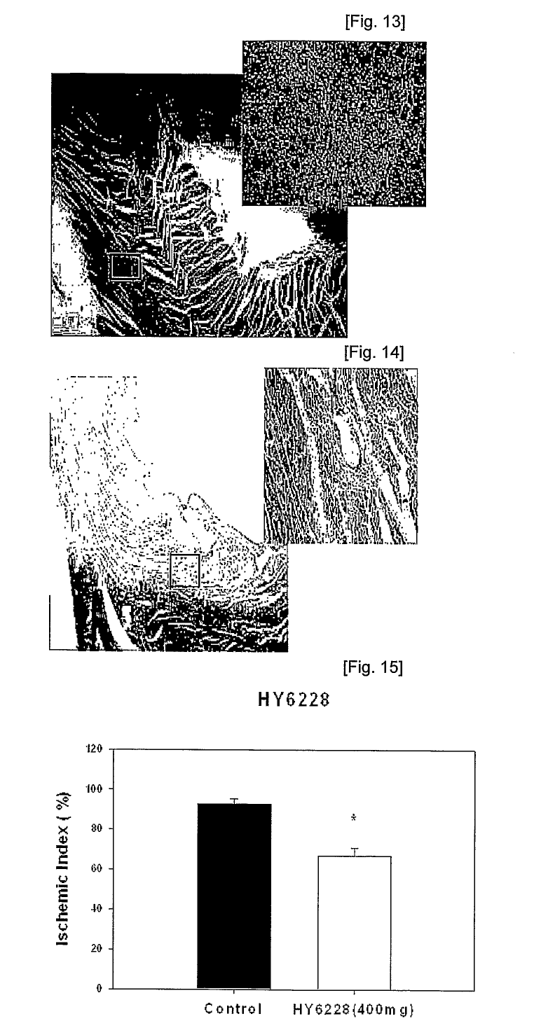

The ischemic index (%) of the HY6228-treated group (n=7) was 67%, while that of the vehicle-treated group (n=12) was 93%, indicating that HY6228 potently reduces the infarct size (28%, p<0.05) (FIG. 15). Therefore, HY6228 is effective in treating cerebral infarction when HY6228 is administered orally.

### EXPERIMENTAL EXAMPLE 7

### Effect of Crude Extract and Butanol Soluble Fraction of Triticum aestivum L. Administered Intraperitoneally on Ischemic Acute Renal Failure (in vivo)

The therapeutic efficacy of HY6228 or H12Bu administered intraperitoneally on ischemic acute renal failure were determined, using animal model in accordance with the procedure cited in the literature (Wang J et al., J. Biol. Chem., 279(19), pp 19948-19954, 2004) with minor modification.

### 7-1. Effect of Crude Extract of Triticum aestivum L. on the Ischemic Acute Renal Failure (in vivo)

SD rats prepared as in Reference Example 1 were anesthetized with 50 mg/kg ketamine (Yuhan corp., KOREA) and 20 mg/kg xylazine (Sigma, USA), and the abdominal cavity was incised. The rats were subjected to bilateral clamping to induce renal ischemia by blocking the blood flow, and then the right renal was eliminated. One hour prior to the clamping of the left renal artery, four-hundred mg/kg of HY6228 dissolved in 1 ml of 0.9% saline solution or the same volume of vehicle was intraperitoneally administered. Forty-five minutes after the ischemia, the clamping was removed for reperfusion. Twenty-four hours after reperfusion, blood samples were collected to measure serum creatinine levels (mg/dl), which is one indication of renal function.

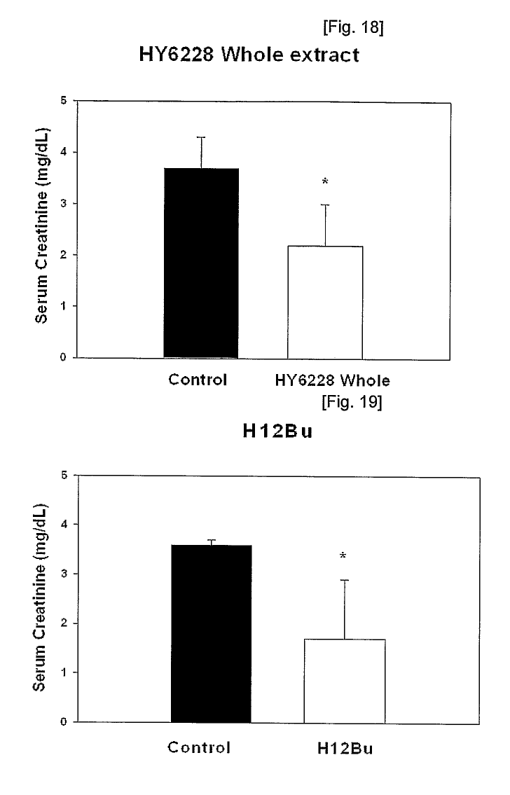

The serum creatinine level of the HY6228-treated group (n=7) was 2.2 mg/dl, while that of the vehicle-treated group (n=4) was 3.7 mg/dl, indicating that HY6228 potently reduces serum creatinine level (40%, p<0.05) (FIG. 18). The results showed that HY6228 reduced the serum creatinine level by promoting excretion of creatinine, which resulted from inhibition of renal injury. Therefore, HY6228 is effective in treating ischemic acute renal failure when HY6228 is administered intraperitoneally.

### 7-2. Effect of Butanol Soluble Fraction Isolated from Triticum aestivum L. Administered Intraperitoneally on Ischemic Acute Renal Failure (in vivo)

The butanol soluble fraction (H12Bu) that was shown, in the above Experimental Example 3, to contain lots of active ingredients was intraperitoneally administered, using the same procedures used in Experimental Example 7-1, as follows;

One hour prior to the clamping of the left renal artery, one-hundred mg/kg of H12Bu dissolved in 1 ml of 0.9% saline solution or the same volume of vehicle was intraperitoneally administered. Forty-five minutes after the ischemia, the clamping was removed for reperfusion. Twenty-four hours after reperfusion, blood samples were collected to measure serum creatinine levels (mg/dl).

The serum creatinine level of the H12Bu-treated group (n=10) was 1.7 mg/dl, while that of the vehicle-treated group (n=6) was 3.6 mg/dl, indicating that H12Bu potently reduces serum creatinine level (53%, p<0.05) (FIG. 19). The results showed that H12Bu also reduced the serum creatinine level by promoting excretion of creatinine. Therefore, H12Bu is also effective in treating ischemic acute renal failure when H12Bu is administered intraperitoneally.

### EXPERIMENTAL EXAMPLE 8

### Effect of Crude Extract of Triticum aestivum L. Administered Orally on Alzheimer's Disease (in vivo)

The therapeutic efficacy of HY6228 orally administered on Alzheimer's disease were determined, using animal model in accordance with the procedure cited in the literature (Yamaguchi Y and Kawashima S, Eur. J. Pharmacol., 412, pp 265-272, 2001), with minor modification.

SD rats prepared as in Reference Example 1 were anesthetized with 50 mg/kg pentobarbital (Hanlim Pharm. Co., Ltd, KOREA) and then placed in a stereotaxic apparatus. The scalp of the region for injection was incised.

And 15 nmol (5 μl)/day of [symbol] amyloid was injected for 14 days in lateral ventricle after lambda and bregma points was located by observation with microscopy. [symbol] amyloid (5 μl) was injected with a syringe pump at a flow rate of 1 μl/min, and the injection needle was left in place for 5 min after injection. For the sham group (Sham), the same procedures were applied, except that physiological saline solution (5 μl) was injected instead of [symbol] amyloid.

After the induction of Alzheimer's disease, the rats were orally treated with 400 mg/kg of HY6228 (HY6228) or physiological saline solution (Control) for 14 days and rested for one week. Water-maze tests were performed in every 24 hours and three tests a day in a row per a rat for 8 days. Water-maze test was performed in a water tank which consists of circular tub (inner dimension: diameter 180 cm, depth 50 cm) filled with clear tap water at the temperature of approximately 22° C., and escape platform (diameter 10 cm, height 25 cm) submerged 2 cm below the surface of the water. The movement of rats was recorded automatically by a video-tracking system (Etho Vision®, Noldus Information Techology, Wageningen, The Netherlands).

In the test, “escape latency” is defined the time taken for the rats to find and escape onto the submerged platform, and then escape latency is accepted only when the rats stay on the platform over 30 s after escaping onto the submerged platform. “Mean escape latency” is average of three escape tests done in a day. Mean escape latency was taken 90 s if the mean escape latency was over 90 s. “Time staying on platform (PF)” was also performed to test whether the rats memorize the location of the platform, and is defined the time for the rats to spend around the location of the platform after the rats arrived at the location, for which experiments the platform was removed in advance. Time staying on PF was measured in every two days and at the third time of the tests done for a rat at the test day (For example: 6^{th} trial on the 2^{nd} day, 12^{th} trial on the 4^{th} day, 18^{th} trial on the 6^{th} day, and 24^{th} trial on the 8^{th} day).

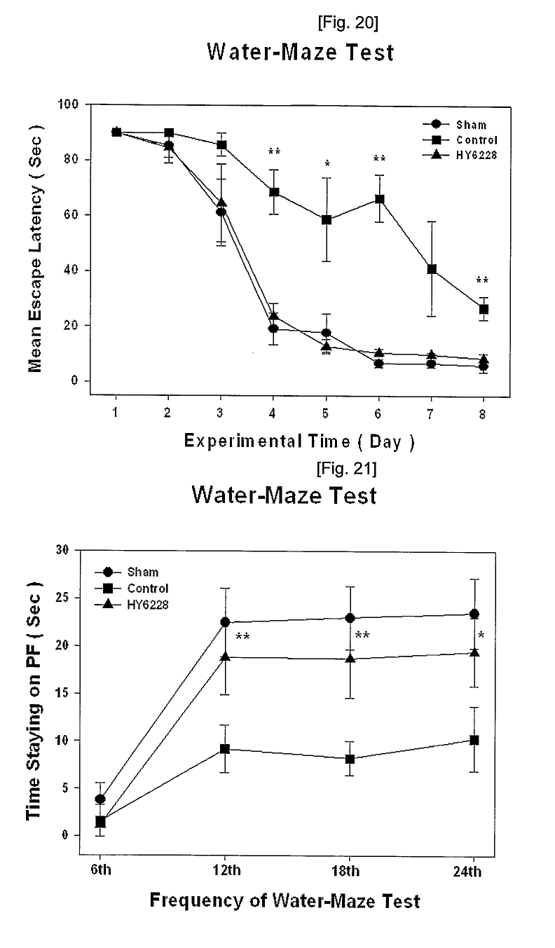

There was a significant decrease in mean escape latency of HY6228-treated group (HY6228) (n=4), compared with that of control group (Control) (n=5) at the 4th, 5th, 6th and 8th day of escape experiments (p<0.01, FIG. 20). There was no significant difference in mean escape latency between HY6228 and Sham groups (n=4). Thus, it was confirmed that the memory loss possibly induced by [symbol] amyloid was prevented, and memory was maintained almost up to the normal level by the treatment with HY6228.

In addition, the staying time on PF of HY6228 group (HY6228) was significantly longer than that of control group (Control) (p<0.01) after the 4th day of the escape experiments i.e. (12^{th} trial on the 4^{th} day; 18^{th} trial on the 6^{th} day; and 24^{th} trial on the 8^{th} day: FIG. 21). These results altogether indicate that intake of HY6228 improves memory not only to find the right direction to the platform, but also to locate the site of the platform. In conclusion, intake of HY6228 improves memory as well as prevents brain damage.

### EXPERIMENTAL EXAMPLE 9

### Toxicity Test

### Methods

The acute toxicity tests on Spague-Dawley rats (mean body weight 320±20 g) were performed using HY6228 of the Example 1. Two groups consisting of 10 rats each were administered with 500 mg/kg by intraperitoneal injection or with 5000 mg/kg orally of HY6228 dissolved in physiological saline solution, respectively, and observed for 24 hours.

### Results

There were no treatment-related adverse effects on mortality, clinical signs, body weight changes or gross findings in any group. These results suggest that the extract prepared in the present invention is potent and safe.

Hereinafter, the formulating methods and kinds of excipient will be described, but the present invention is not limited to them. The representative preparation examples were described as follows.

### Preparation of Powder

| | |
|---|---|
| HY6228 of Example 1 | 50 mg |
| Lactose | 100 mg |
| Talc | 10 mg |

Powder preparation was prepared by mixing the components described above, and by filling in sealed package.

### Preparation of Tablet

| | |
|---|---|
| HY6228 of Example 1 | 50 mg |
| Corn Starch | 100 mg |
| Lactose | 100 mg |
| Magnesium Stearate | 2 mg |

Tablet preparation was prepared by mixing the components described above, and by entabletting them.

### Preparation of Capsule

| | |
|---|---|
| HY6228 of Example 1 | 50 mg |
| Corn starch | 100 mg |
| Lactose | 100 mg |
| Magnesium Stearate | 2 mg |

Tablet preparation was prepared by mixing the components described above, and by filling gelatin capsule by conventional gelatin preparation method.

### Preparation of Injectables

| | |
|---|---|
| HY6228 of Example 1 | 50 mg |
| Distilled water for injection | optimum amount |
| PH controller | optimum |
| | amount |

Injection preparation was prepared by dissolving active component, by controlling pH to about 7.5, by filling all the components in 2 ml ample, and then by sterilizing by conventional injection preparation method

### Preparation of Liquid

| | |
|---|---|
| HY6228 of Example 1 | 0.1~80 g |
| Sugar | 5~10 g |
| Citric acid | 0.05~0.3% |
| Caramel | 0.005~0.02% |
| Vitamin C | 0.1~1% |
| Distilled water | 79~94% |
| CO₂ | 0.5~0.82% |
| gas | |

Liquid preparation was prepared first by dissolving the active component in distilled water, by filling all the components, and by sterilizing by conventional liquid preparation method.

### Preparation of Health Food

| HY6228 of Example 1 | 1000 | mg |
|---|---|---|
| Vitamin mixture | optimum amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | g |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in many ways. Such variations are not to be regarded as a departure from the spirit and scope of the present invention.

### Preparation of Health Beverage

| | | |
|---|---|---|
| HY6228 of Example 1 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirring at 85° C. for 1 hour, filtering and then filling all the components in 1000 ml ample and sterilizing by conventional health beverage preparation method

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the extracts of Gramineae plant of the present invention reduce the infarcted regions by inhibiting cell apoptosis on ischemic animal model. The invented extract prevents heart and brain damages and improves memory in Alzheimer's animal model with no side effects. Therefore, it can be used as a therapeutics or health care food for treating and preventing ischemic diseases or degenerative brain diseases.

1. A pharmaceutical composition comprising crude extract or purified fraction of Gramineae plant as an active ingredient and pharmaceutically acceptable carriers or adjuvants for the treatment and prevention of ischemic diseases.

2. The pharmaceutical composition according to claim 1, wherein said ischemic diseases comprise various ischemic diseases such as myocardial infarction, cerebral infarction, ischemic acute renal failure, ischemic acute hepatic failure, diabetic foot ulcer, diabetic nephropathy, and ischemic diseases or organ/tissue injury that result from side effect of surgical operation.

3. The pharmaceutical composition according to claim 2, wherein said ischemic diseases resulting from side effect of surgical operation comprise ischemic heart failure, ischemic renal failure, ischemic hepatic failure or ischemic stroke.

4. The pharmaceutical composition according to claim 2, wherein said organ/tissue injury resulting from side effect of surgical operation is caused by ischemia-reperfusion when organ surgery and transplantation, or reconnection of parts of body severed by an accident is performed.

5. The pharmaceutical composition according to claim 4, wherein said organ comprises internal organs, for example, kidney, liver, pancreas, lung or heart, and external organs, such as leg, hand, finger, or ear.

6. A use of crude extract or purified fraction of Gramineae plant for the preparation of therapeutic agent for the prevention and treatment of ischemic diseases caused by apoptosis in a mammal.

7. A pharmaceutical composition comprising crude extract or purified fraction of Gramineae plant as an active ingredient and pharmaceutically acceptable carriers or adjuvants for the prevention and treatment of degenerative brain diseases.

8. The pharmaceutical composition according to claim 7, wherein said degenerative brain diseases comprise various degenerative brain diseases such as dementia of the Alzheimer's type (Alzheimer's disease), vascular dementia, Parkinson's disease, amyotrophic lateral sclerosis, Huntington's disease, Pick's disease, Creutzfeldt-Jakob disease or spinal cord injury.

9. The pharmaceutical composition according to claim 1, wherein said Gramineae plant is selected from the group consisting of Triticum aestivum L., floating Triticum aestivum L., Secale cereale L., brown rice, Horden vulgare L., malt, Avena sativa L., Zea mays L., Sorghum bicolor MOENCH, Coix lacryma-jobi var. mayuen STAPF, Panicum miliaceum L. or Setaria italica Beauv.

10. The pharmaceutical composition according to claim 1, wherein said crude extract is extracted with the solvent selected from the group consisting of water, lower alcohol and the mixture thereof.

11. The pharmaceutical composition according to claim 1, wherein said purified fraction comprises the purified fractions isolated from Gramineae plant prepared by the process comprising the following steps;
step 1, adjusting the crude extract of Gramineae plant to pH 7 and pH 12 by adding strong base;
step 2, removing the non-polar substance in the crude extract of Gramineae plant by adding equivalent amount of non-polar solvent to the extract prepared in step 1, to obtain water-soluble extract thereof;
step 3, subjecting to extraction and fractionation of the water soluble extract, prepared in step 2, by adding equivalent amount of lower alcohol thereto to obtain the butanol-soluble fraction, and water soluble fraction isolated from the extract prepared in pH 12; butanol-soluble fraction, and water soluble fractions isolated from the extract prepared in pH 7 of the present invention.

12. The pharmaceutical composition according to claim 11, wherein said purified fraction is butanol-soluble fraction isolated from the extract prepared in pH 12.

13. A use of crude extract or purified fraction of Gramineae plant for the preparation of therapeutic agent for the prevention and the treatment of degenerative brain diseases caused by apoptosis in mammal and human.

14. A method for preparing the purified fraction isolated from Gramineae plant comprising the steps consisting of the following steps;
step 1, adjusting the a crude extract of Gramineae plant of claim 1 to pH 7 and pH 12 by adding strong base;
step 2, removing non-polar substance in the crude extract of Gramineae plant by adding equivalent amount of non-polar solvent to the extract prepared in step 1, to obtain water-soluble extract thereof;
step 3, subjecting to extraction and fractionation of the water soluble extract, prepared in step 2, by adding equivalent amount of lower alcohol thereto to obtain the butanol-soluble fraction, and water soluble fraction isolated from the extract prepared in pH 12; butanol-soluble fraction, and water soluble fractions isolated from the extract prepared in pH 7 of the present invention.

15. A health care food comprising crude extract or purified fraction of Gramineae plant as an active ingredient, together with a sitologically acceptable additive for the prevention and improvement of ischemic diseases or degenerative brain diseases caused by apoptosis.

16. The health care food according to claim 15, wherein said health care food is selected from the group consisting of powder, granule, tablet, chewing tablet, capsule and beverage type.

17. The use of crude extract or purified fraction of Gramineae plant of claim 6, wherein the mammal is a Human.

18. The pharmaceutical composition according to claim 7, wherein said Gramineae plant is selected from the group consisting of Triticum aestivum L., floating Triticum aestivum L., Secale cereale L., brown rice, Horden vulgare L., malt, Avena sativa L., Zea mays L., Sorghum bicolor MOENCH, Coix lacryma-jobi var. mayuen STAPF, Panicum miliaceum L. and Setaria italica Beauv.

19. The pharmaceutical composition according to claim 7, wherein said crude extract is extracted with the solvent selected from the group consisting of water, lower alcohol and the mixture thereof.

20. The pharmaceutical composition according to claim 7, wherein said purified fraction comprises the purified fractions isolated from Gramineae plant prepared by the process comprising the following steps;
step 1, adjusting the crude extract of Gramineae plant to pH 7 and pH 12 by adding strong base;
step 2, removing the non-polar substance in the crude extract of Gramineae plant by adding equivalent amount of non-polar solvent to the extract prepared in step 1, to obtain water-soluble extract thereof;
step 3, subjecting to extraction and fractionation of the water soluble extract, prepared in step 2, by adding equivalent amount of lower alcohol thereto to obtain the butanol-soluble fraction, and water soluble fraction isolated from the extract prepared in pH 12; butanol-soluble fraction, and water soluble fractions isolated from the extract prepared in pH 7 of the present invention.
